(19) United States
(12) Patent Application Publication
(10) Pub. No.: US 2022/0011870 A1
(43) Pub. Date: Jan. 13, 2022

(54) TOUCHLESS CONTROL PANEL

(71) Applicant: Royal Caribbean Cruises Ltd., Miami, FL (US)

(72) Inventors: Joshua Takashi Nakaya, Miami, FL (US); Joey Hasty, Winter Park, FL (US); Anibal Damian Sarno, Miami, FL (US); Ganna Radzevych, Miami, FL (US)

(21) Appl. No.: 16/923,995

(22) Filed: Jul. 8, 2020

(51) Int. Cl. G06F 3/01; G06F 3/03; A61L 2/10; A61L 2/24

(52) U.S. Cl. CPC G06F 3/017; G06F 3/016; A61L 2/24; A61L 2/10; G06F 3/0304

(57) ABSTRACT

Touchless control panel systems and methods are disclosed. A touchless control panel may include a housing and one or more user inputs. The individual user inputs can include an aperture extending through the housing, a primary sensor disposed to detect objects inserted a first distance into the housing through the aperture and to generate a first signal when an object is detected, and a haptic device configured to provide haptic feedback to objects inserted through the aperture. Processing circuitry in communication with the one or more user inputs can be configured to, for each of the one or more user inputs, receive the first signal from the primary sensor, transmit a control signal based on the first signal, and cause the haptic device to provide haptic feedback based on the first signal. The disclosed touchless control panels may be installed as elevator control stations.

### BACKGROUND

### Technological Field

The present application relates to touchless control panel systems and methods for preventing the spread of contaminants.

### Description of the Related Art

A fomite is an inanimate object that, when contaminated with or exposed to infectious agents, can transfer disease to a new host. Shared surfaces in public spaces are common fomites that present a risk of transfer of contaminants, such as bacteria, viruses, fungi, or other pathogenic or toxic agents that can be spread from person to person. Examples of common fomites include, for example, elevator control stations, elevator call buttons, building intercoms, public telephone controls, pedestrian crossing buttons, vending machines, parking payment or ticket machines, bus stop request buttons, and the like. Surfaces including push buttons are especially suitable for contaminant transfer, as many repeated user contacts may be concentrated at the push button locations rather than being randomly distributed over a surface. After a user depresses a push button and leaves an infectious substance (e.g., infected bodily fluids, skin cells, hair, etc.) on the push button, an infectious agent in the substance may subsequently be transferred to many others who later use the same push button. This is a particularly acute problem in high traffic areas where users are contacting a surface within intervals (such as seconds, minutes, or hours) during which many infectious agent remain capable of transferring disease. Moreover, the spread of infection via common surfaces can make contact tracing more difficult in tracking the spread of a disease due to the anonymous nature of the transfer. Accordingly, it may be desirable to replace push buttons in some implementations with control devices that do not require physical touching of a surface to actuate a control signal.

### SUMMARY

The system, method, and devices of this disclosure each have several innovative aspects, no single one of which is solely responsible for its desirable attributes disclosed herein. Without limiting the scope of this disclosure, its more prominent features will now be discussed briefly. After considering this discussion, and particularly after reading the section entitled “Detailed Description” one will understand how the features of this disclosure provide advantages over other personalized recommendation solutions.

In a first aspect, a touchless control panel is described. The touchless control panel includes a housing, one or more user inputs, and processing circuitry in communication with the one or more user inputs. Each of the one or more user inputs includes an aperture extending through the housing, a primary sensor disposed to detect objects inserted a first distance into the housing through the aperture and to generate a first signal when an object is detected, and a haptic device configured to provide haptic feedback to objects inserted through the aperture. The processing circuitry is configured to, for each of the one or more user inputs, receive the first signal from the primary sensor, transmit a control signal based on the first signal, and cause the haptic device to provide haptic feedback based on the first signal.

In some embodiments, the haptic device includes at least one of an ultrasound emitter or an air emitter at least partially disposed within the housing.

In some embodiments, each of the one or more user inputs further includes a secondary sensor disposed to detect objects inserted a second distance greater than the first distance into the housing through the aperture and to generate a second signal when an object is detected, and the processing circuitry is further configured to, for each of the one or more user inputs, receive the second signal from the secondary sensor, and cause the haptic device to provide an increased level of haptic feedback in response to the second signal. In some embodiments, the processing circuitry is further configured to cause the haptic device to discontinue the increased level of haptic feedback in response to a non-triggered state of the secondary sensor. In some embodiments, discontinuing the increased level of haptic feedback comprises returning to a reduced level of haptic feedback. In some embodiments, discontinuing the increased level of haptic feedback comprises deactivating the haptic device.

In some embodiments, the primary sensor comprises a photoelectric sensor configured to emit a light beam across a portion of an interior of the housing proximate the aperture and to generate the first signal when the light beam is interrupted.

In some embodiments, each of the one or more user inputs further includes a light source disposed to irradiate at least a portion of the housing with germicidal radiation. In some embodiments, the light germicidal radiation includes ultraviolet light having a wavelength in a far-ultraviolet C wavelength range between about 207 nm and about 222 nm. In some embodiments, the light source includes a ring extending along a lateral surface of the aperture, the ring comprising at least one visible light-emitting diode (LED) configured to emit visible light and at least one ultraviolet LED configured to emit far-ultraviolet C germicidal radiation. In some embodiments, the at least one ultraviolet LED is configured to operate continuously, and the processing circuitry activates the at least one visible LED in response to the first signal.

In some embodiments, the touchless control panel includes at least one of an elevator control station or an elevator call button panel, and the processing circuitry transmits the control signal to an elevator controller remote from the touchless control panel.

In a second aspect, a method of operating a touchless control panel is described. The method includes detecting an object at a first proximity sensor disposed within a housing of a touchless control panel and, in response to detecting the object, causing a haptic device within the housing to provide haptic feedback to the object and transmitting a control signal corresponding to a control function associated with the first proximity sensor.

In some embodiments, causing the haptic device to provide haptic feedback comprises causing the haptic device to emit at least one of air or ultrasound.

In some embodiments, the method further includes detecting the object at a second proximity sensor disposed within the housing and in response to detecting the object at the second proximity sensor, causing the haptic device to increase an intensity or a frequency of the haptic feedback. In some embodiments, the method further includes detecting, at the second proximity sensor, that the object has been at least partially removed and, in response to detecting that the object has been at least partially removed, discontinuing the increased intensity or frequency of the haptic feedback. In some embodiments, discontinuing the increased intensity or feedback includes returning to a primary intensity or frequency until the object is removed from the housing. In some embodiments, discontinuing the increased intensity or feedback includes deactivating the haptic device.

In some embodiments, the method further includes, in response to detecting the object, providing a visual indication associated with the control function.

In some embodiments, the control signal causes, at least in part, an elevator controller to control an elevator to implement the control function.

### BRIEF DESCRIPTION OF THE DRAWINGS

The disclosed aspects will hereinafter be described in conjunction with the appended drawings and appendices, provided to illustrate and not to limit the disclosed aspects, wherein like designations denote like elements.

FIG. 1 illustrates an interior or exterior portion of an elevator having a touchless control panel installed thereon in accordance with the present technology.

FIGS. 2A and 2B illustrate an example touchless control panel in accordance with the present technology.

FIGS. 3A-3D illustrate an example operational sequence of the touchless control panel of FIGS. 2A and 2B.

FIG. 4 illustrates disinfecting features of a touchless control panel in accordance with the present technology.

FIG. 5 schematically illustrates electronic components of an example touchless control panel in accordance with the present technology.

FIG. 6 is a flowchart illustrating an example method of operating a touchless control panel in accordance with the present technology.

### DETAILED DESCRIPTION

Pushbuttons are frequently encountered in everyday life. Calling or controlling an elevator, crossing the street at a crosswalk, requesting a bus stop, and entering a parking garage, for example, are among the many activities that frequently involve depressing a button that is shared among many other users. Such contacts can thus present a risk of transmission of infectious agents or other contaminants. Accordingly, it is desirable to provide a system capable of receiving similar inputs without requiring users to touch a physical button.

Conventional pushbuttons inherently provide a confirmation to a user that they have been pressed, as the user receives a tactile sensation of motion and/or resistance as the button is depressed. The user may also feel the pushbutton hit a stop at the end of its range of motion. Compensating for the lack of such immediate tactile feedback is just one challenge of implementing a functional touchless control panel.

The present technology provides touchless control panel systems and methods that allow a user to provide a control input without touching any shared surfaces, thereby reducing the probability of the control panel transferring disease among its users. In example embodiments described herein, a touchless control panel can include a housing containing one or more, or an array, of user inputs. Each user input may appear from the exterior as an opening in a faceplate of the housing, optionally accompanied by a label adjacent to the opening indicating a control function associated with the user input (e.g., a floor number or other function frequently found on elevator control station buttons). Within the housing, a proximity sensor and a haptic device capable of producing touchless haptic feedback are included for and aligned with each user input opening. The proximity sensor may be, for example, a photoelectric sensor disposed such that a light beam of the sensor crosses a space in the interior of the housing behind the opening. The haptic device can produce touchless haptic feedback using, for example, a puff or vortex of air, or ultrasound (e.g., an ultrasound beam) to induce a feeling of touch. Thus, when a user inserts a finger through the opening of a user input, the interruption of the light beam triggers both the associated control function and contactless haptic feedback that induces a sensation in the user's finger to confirm that the input has been received.

In order to prevent inadvertent touching of the interior of the touchless control panel, in some embodiments the individual user inputs can include a secondary proximity sensor aligned with the opening, but disposed farther into the interior of the housing and closer to the haptic device. For example, the secondary proximity sensor may be disposed at a depth at which the user's finger is at risk of contacting the surface of the haptic device or an interior surface of the housing. If the user's finger moves far enough into the opening to interrupt a beam of the secondary proximity sensor, a more aggressive haptic feedback can be triggered at the haptic device. The more aggressive haptic feedback can indicate to the user that the finger should be removed.

In some cases, incidental contact may still occur in some areas, such as around the edges of the openings. Such incidental contacts may be mitigated by further including a human-safe germicidal illumination source (for example, but not limited to, a far-UVC light source) that continuously or periodically emits light to inactivate pathogens, such as bacteria or viruses, that may have been deposited by a previous incidental contact and remain on the contacted surface(s). For example, the sides of the openings may be lined with a light ring that includes one or more far-UVC LED light sources.

Thus, the systems and methods of the present technology provide touchless control panels that do not require contacting a surface to provide an input, and may even actively prevent contacting the surfaces of the control panel and/or disinfect surfaces that may receive occasional unintended contact. Moreover, the disclosed touchless control panels include haptic and other feedback features that provide a tactile experience as intuitive as that of conventional pushbutton control panels.

Although example embodiments of the present technology are described with reference to control of an elevator, it will be understood that the present technology may be implemented in any context in which a pushbutton may otherwise be implemented. For example, the present technology may be implemented in elevator control stations, elevator call buttons, building intercoms, public telephone controls, pedestrian crossing buttons, vending machines, parking payment or ticket machines, bus stop request buttons, or any other locations in which touchless switch activation or communication is desirable.

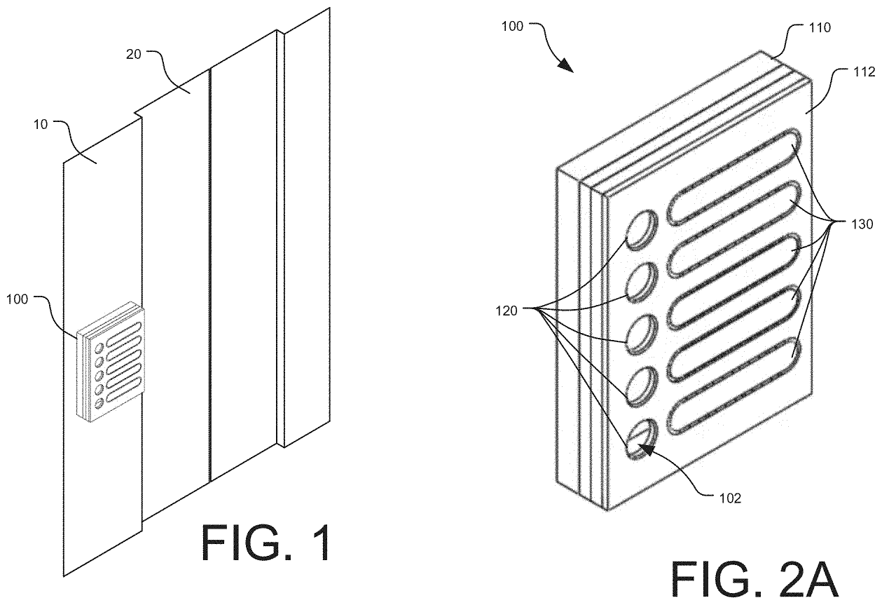

FIG. 1 illustrates one particular example implementation of a touchless control panel 100 in accordance with the present technology. As shown in FIG. 1, a touchless control panel 100 is installed on an interior or exterior wall 10 of an elevator adjacent to interior or exterior doors 20 of the elevator. If installed in an interior of the elevator, the touchless control panel 100 may serve as an elevator control station including various controls such as floor buttons, door open and close buttons, etc. If installed in an exterior of the elevator, the touchless control panel 100 may function as an elevator call button panel, for example, including up and down call buttons, etc. As will be described in greater detail, the touchless control panel 100 is configured to receive contactless input from users, to activate control functions based on the input, and to provide haptic, audio, and/or visual feedback responsive to the input.

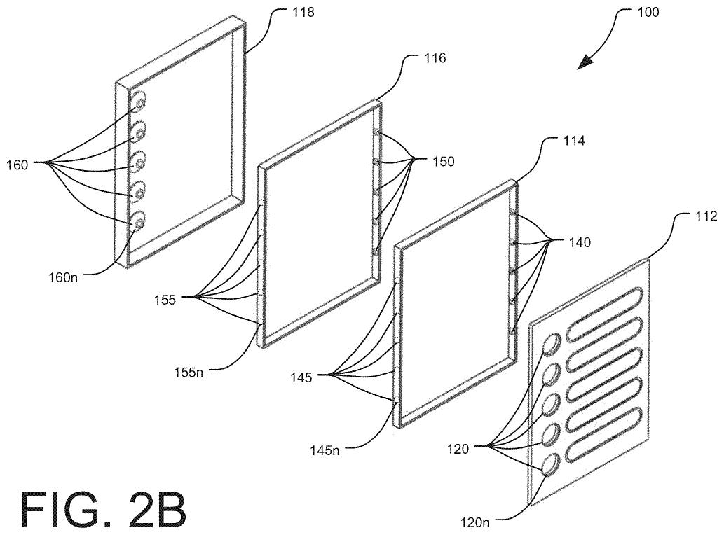

FIGS. 2A and 2B illustrate an example touchless control panel 100 in accordance with the present technology. FIG. 2A is a perspective view of the exterior of the touchless control panel 100. FIG. 2B is an exploded view illustrating exterior and interior components of the touchless control panel 100. The embodiment of the touchless control panel 100 illustrated in FIGS. 2A and 2B is exemplary, and various other embodiments may include fewer than all of the components illustrated herein and/or may contain additional components not illustrated herein, without departing from the spirit or scope of the present technology.

The touchless control panel 100 includes a housing 110 at least partially surrounding an interior space 102. A faceplate 112 forms an outward-facing portion of the housing 110. The faceplate 112 includes one or more apertures 120 extending through the faceplate 112 between the exterior and the interior 102 of the housing 110. The apertures 120 have a suitable diameter so as to be sized and shaped to each accommodate a human finger therethrough. For example, the apertures 120 may have a diameter between about 0.5 inches and about 3 inches, between about 1 inch and about 2 inches, or within any other suitable subrange. In some embodiments, the diameter of each aperture 120 is approximately 1 inch, approximately 1.25 inches, approximately 1.5 inches, approximately 1.75 inches, or approximately 2 inches. It will be understood that embodiments of the present technology are not limited to an aperture 120 sized and shaped to accommodate a human finger. In another non-limiting example, an aperture 120 is sized and shaped to accommodate an inanimate implement, such as a stylus, a rod, a tool, a writing implement, or any other object having a distal end and a proximate end arranged along a longitudinal axis.

As shown in the exploded view of FIG. 2B, the housing 110 further includes a primary sensor array housing 114, a secondary sensor array housing 116, and a haptic device array housing 118. In various embodiments, some or all of the faceplate 112, the primary sensor array housing 114, the secondary sensor array housing 116, and the haptic device array housing 118 may be combined as a single integrally formed housing or housing component.

Within the housing 110, the touchless control panel 110 includes one or more primary sensors 140/145, one or more secondary sensors 150/155, and one or more haptic devices 160. In the example embodiment of the touchless control panel 100, each primary sensor 140/145 is a photoelectric sensor including a transmitter 140 configured to emit a beam of light (e.g. a laser beam) and a receiver 145 positioned to detect the beam emitted by the transmitter 140. Similarly, each secondary sensor 150/155 is a photoelectric sensor including a transmitter 150 configured to emit a beam of light (e.g. a laser beam) and a receiver 155 positioned to detect the beam emitted by the transmitter 150. Each of the primary sensors 140/145 and the secondary sensors 150/155 may be configured to generate a signal when the corresponding beam is interrupted, such as by an object crossing a plane that includes the beam and is generally perpendicular to the direction of motion of the object or generally parallel to the faceplate 112. Other types of proximity sensors may be suitably implemented as the primary sensors 140/145 and/or the secondary sensors 150/155 (e.g., capacitive sensors, other optical sensors, etc.). In some embodiments, the primary sensors 140/145 are disposed to detect objects inserted a first distance into the housing 110 through the apertures 120, and the secondary sensors 150/155 are disposed to detect objects inserted a second distance greater than the first distance into the housing 110 through the apertures 120.

The haptic devices 160 may be any suitable device for providing non-contact haptic feedback. In one example, the haptic devices 160 are compressed air haptic devices which emit puffs of air and/or air vortex rings to provide haptic feedback to an object such as a finger that is not in physical contact with the haptic device 160. As will be explained below, proximity of the object as it moves closer to the haptic device 160 can trigger the non-contact haptic feedback when the object reaches a predetermined distance from the haptic device 160. In another example, the haptic devices 160 are ultrasonic haptic devices which emit ultrasound, such as in one or more focused ultrasound beams, to create a localized sense of pressure on an object such as a finger that is not in physical contact with the haptic device 160. Each ultrasound haptic device may be an array of ultrasound emitters. In some embodiments, each haptic device 160 may include two or more individual devices such as ultrasound emitters or air emitters, or a combination thereof.

In some embodiments, the haptic devices 160 can be configured to have at least three operational states including at least two different levels of haptic feedback. For example, each haptic device 160 can have a standby state in which it does not produce haptic feedback, a primary haptic feedback state in which it produces a first level of haptic feedback, and a secondary haptic feedback state in which it produces a second level of haptic feedback different from the first level of haptic feedback. The first level of haptic feedback may be, for example, emission of air or ultrasound to produce a first sensation. The second level of haptic feedback may have a different intensity (e.g., stronger or weaker) than the first sensation, may have a different frequency than the first sensation, or may otherwise be configured to induce a different haptic sensation than the first sensation. In some embodiments, the secondary haptic feedback state may be activated by modifying the operation of a single haptic device 160, or by activating a plurality of devices within the haptic device 160.

The touchless control panel 100 thus includes one or more user inputs that perform at least the functions conventionally performed by pushbuttons. In one example, each user input includes one aperture 120, one primary sensor 140/145 aligned with the aperture 120, one secondary sensor 150/155 aligned with the aperture and the primary sensor 140/145, and one haptic device 160 aligned with the aperture 120, the primary sensor 140/145, and the secondary sensor 150/155. For example, in the example touchless control panel 100, one example “user input” includes the bottommost aperture 120n, the primary sensor 140n/145n vertically aligned with the aperture 120n, the secondary sensor 150n/155n vertically aligned with the aperture 120n and the primary sensor 140n/145n, and the haptic device 160n vertically aligned with the aperture 120n, the primary sensor 140n/145n, and the secondary sensor 150n/155n. In some embodiments, the individual user inputs can include a single sensor 140/145 and may not include a secondary sensor 150/155 (e.g., if only a single haptic feedback state is used).

Each user input may include or be located adjacent to a user input label 130 indicating a function associate with the user input. Examples of user input labels 130 include, but are not limited to, elevator floor number indicators (e.g., alphanumeric indicators of building floors, ship decks, parking levels, etc.), elevator call button indicators (e.g., “UP” or “DOWN,” or corresponding symbols), or any other suitable label depending on the functionality of the user inputs.

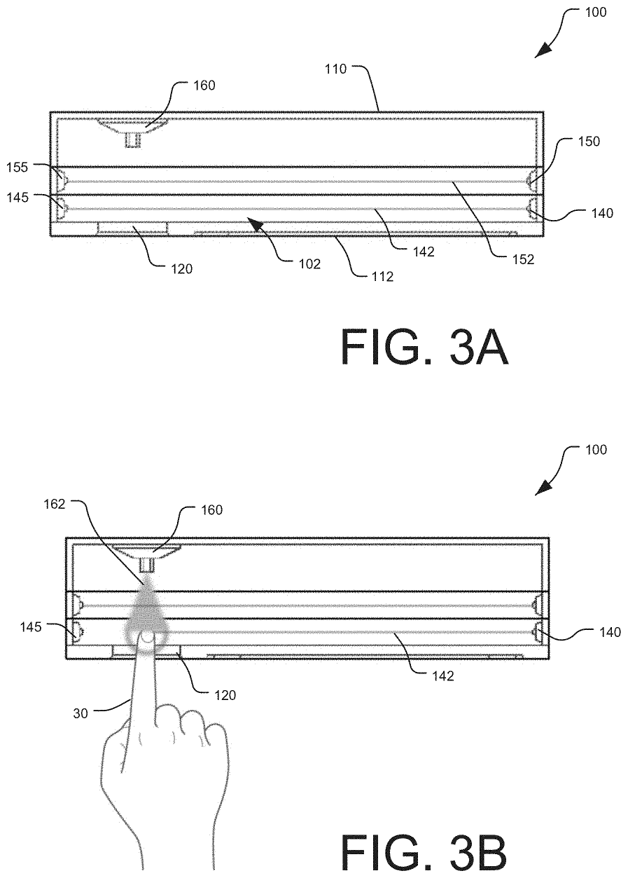

Referring now to FIGS. 3A-3D, an example operation of the touchless control panel 100 will now be described. FIGS. 3A-3D depict a cross-sectional top view of the touchless control panel 100 described with reference to FIGS. 2A and 2B. The cross-sectional top view of FIGS. 3A-3D includes the housing 110 and the components of a single one of the one or more user inputs. The user input includes an aperture 120 extending through the faceplate 112, as well as a corresponding primary sensor 140/145, secondary sensor 150/155, and haptic device 160 disposed within the interior 102 of the housing 110.

The example operation of the touchless control panel 100 begins at FIG. 3A. The touchless control panel 100 in FIG. 3A is ready to receive an input from a user. In the ready configuration of FIG. 3A, the primary sensor 140/145 and the secondary sensor 150/155 are active so as to detect objects inserted at the aperture 120. For example, the transmitter 140 of the primary sensor 140/145 emits a beam 142 which is detected by the receiver 145. Similarly, the transmitter 150 of the secondary sensor 150/155 emits a beam 152 which is detected by the receiver 155. Alternatively, in some embodiments only the primary sensor 140/145 is active, and the secondary sensor 150/155 becomes active (e.g., the transmitter 150 begins emitting the beam 152) based on the detection of an object by the primary sensor 140/145. The beams 142, 152 can be any type of beam whose interruption or disruption by an object can be detected. In this non-limiting example, the beams 142, 152 are laser beams having an intensity and wavelength that are not harmful to human tissues. Other types of human-safe detector beams may similarly be implemented.

At FIG. 3B, a user inserts a finger 30 into the user input of the touchless control panel 100 through the aperture 120. When the finger 30 crosses the path of the beam 142 of the primary sensor 140/145, the finger 30 interrupts the beam 142. As the beam 142 is interrupted, the primary sensor 140/145 is triggered when the receiver 145 ceases receiving the beam 142. In response, the primary sensor 140/145 (e.g., the transmitter 140, the receiver 145, or other processing circuitry in communication with the transmitter 140 or the receiver 145) generates or transmits a signal to a controller or other processing circuitry of the touchless control panel 100 indicating that the primary sensor 140/145 of the user input has been triggered. In response to the signal, the haptic device 160 provides primary haptic feedback 162 (e.g., haptic feedback at a first intensity level) in the form of a puff or vortex of air or an ultrasound beam directed toward the finger 30. In addition, the appropriate control signal (e.g., triggering a floor selection or other control operation associated with the user input) is generated or transmitted in response to the signal from the primary sensor 140/145. In some embodiments, a visual indication of the received input may be provided to the user. For example, one or more lights around the aperture 120 may illuminate or a visual acknowledgement may be displayed on a display located on or near the touchless control panel 100.

Based on the haptic feedback 162 and/or based on the optional visual indication of a successful user input, the user may withdraw the finger 30 from the aperture 120. However, in some instances a user may insert the finger 30 farther into the aperture 120 along the axis along which the haptic device 160 and the aperture 120 are generally aligned. In such instances, it may be desirable to prevent the user's finger 30 from continuing to move toward the haptic device 160 along the axis and potentially touching interior surfaces of the touchless control panel 100. As shown in FIG. 3C, as the user continues inserting the finger 30, the finger 30 crosses the path of the beam 152 of the secondary sensor 150/155. As the beam 152 is interrupted, the secondary sensor 150/155 is triggered when the receiver 155 ceases receiving the beam 152. In response, the secondary sensor 150/155 (e.g., the transmitter 150, the receiver 155, or other processing circuitry in communication with the transmitter 150 or the receiver 155) generates or transmits a signal to a controller or other processing circuitry of the touchless control panel 100 indicating that the secondary sensor 150/155 of the user input has been triggered.

In response to the signal, as shown in FIG. 3D, the haptic device 160 provides secondary haptic feedback 164 (e.g., haptic feedback at a second intensity level greater than the first intensity level) in the form of a puff or vortex of air or an ultrasound beam directed toward the finger 30. In some embodiments, the secondary haptic feedback 164 may be a higher intensity or frequency of the same type of haptic feedback as the primary haptic feedback 162. For example, the primary haptic feedback 162 of FIG. 3B may be provided at an intensity or frequency selected to be high enough to be detectable but not to be uncomfortable. The secondary haptic feedback 164 may be provided at a more aggressive intensity or frequency, which may be selected to induce mild discomfort or sufficiently increased resistance to indicate to the user that the finger 30 should not be inserted further into the aperture 120 and/or to induce the user to involuntarily withdraw the finger 30. In some embodiments, the secondary haptic feedback 164 can be of a different type than the primary haptic feedback 162. For example, in some embodiments the primary haptic feedback 162 may be a puff or vortex of air, and the secondary haptic feedback 164 can be ultrasound, or vice versa. Thus, by the operational process illustrated in FIGS. 3A-3D, the touchless control panel 100 is capable of receiving a touchless control input from a user and preventing the user from touching any interior surfaces or the haptic device 160.

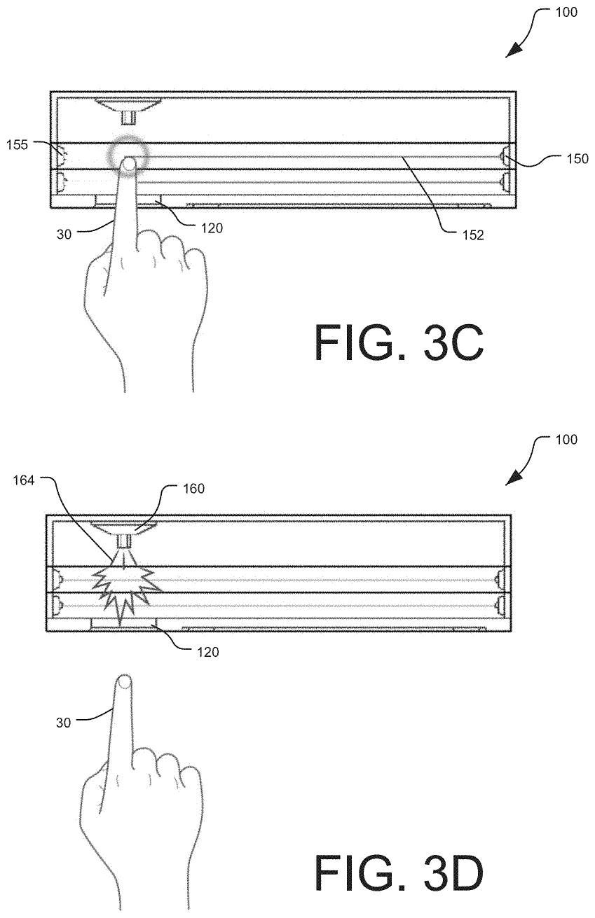

Although the haptic device 160 can prevent most users from touching the interior surfaces of the touchless control panel 100, it may be difficult to prevent occasional contact between a finger 30 and the lateral sides of the apertures 120. Referring now to FIG. 4, the touchless control panel 100 may further include one or more disinfecting features to avoid the transmission of infectious agents via the surfaces of the aperture 120 and/or via the interior surfaces of the touchless control panel 100. In some embodiments, one or more sterilizing light sources 122 may be provided within the lateral sides of the apertures 120. In one example implementation, the sterilizing light sources 122 may be disposed within a ring structure extending along at least a portion of the lateral surface of each aperture 120.

In some embodiments, the sterilizing light sources 122 include light emitting diodes (LEDs) configured to emit one or more wavelengths of germicidal ultraviolet (UV) light. The germicidal UV light may be, for example, light within the UVC range of about 100 nm to about 280 nm. Preferably, the germicidal UV light is far-UVC light within the range of about 207 nm to about 222 nm, as far-UVC light has been determined to be capable of inactivating pathogens such as bacteria, viruses, and protozoa without harming exposed human cells or tissues.

As discussed above, some embodiments of the touchless control panel 100 are capable of providing a visual indication to a user by illuminating the exterior of the aperture 120 when an input is received. In such embodiments, a light ring structure disposed along the lateral sides of each aperture 120 may include a combination of both a visible light source (e.g., visible LEDs configured to emit light in one more visible wavelengths) and a sterilizing light source 122 (e.g., far-UVC LEDs configured to emit light in one or more human-safe, germicidal far-UVC wavelengths). In some embodiments, the light rings are controlled such that the sterilizing light sources 122 are continuously illuminated, and the individual visible light sources are only illuminated intermittently to serve as a visual indicator when a user input is received at the corresponding aperture 120.

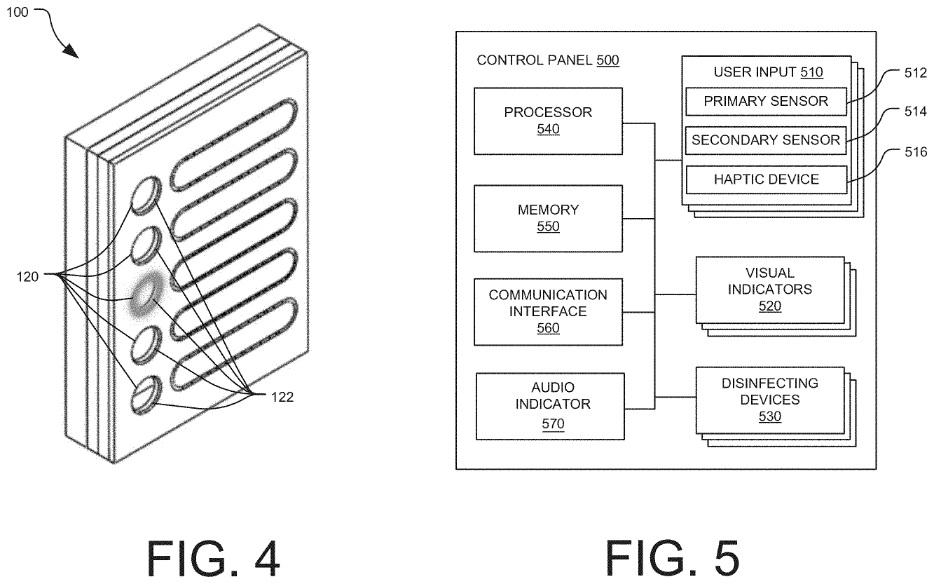

FIG. 5 schematically illustrates electronic components of an example touchless control panel 500 in accordance with the present technology. The touchless control panel 500 may be, for example, the touchless control panel 100 of FIGS. 1-4, or may be any other embodiment of a touchless control panel in accordance with the present technology. The embodiment of the touchless control panel 500 illustrated in FIG. 5 is exemplary, and various other embodiments may include fewer than all of the components illustrated herein and/or may contain additional components not illustrated herein, without departing from the spirit or scope of the present technology.

The touchless control panel 500 includes one or more user inputs 510, one or more visual indicators 520, one or more disinfecting devices 530, a processor 540, a memory 550, a communication interface 560, and an audio indicator 570.

Each user input 510 may correspond to the functionality of a button in a conventional pushbutton control panel, and includes a primary sensor 512, a secondary sensor 514, and a haptic device 516. Each user input 510 may be, for example, a user input as illustrated and described with reference to FIGS. 3A-3D. Each user input 510 is at least configured to detect an object inserted into the user input 510 at the primary sensor 512, to transmit a signal to the processor 540 when an object is detected at the primary sensor 512, and to cause the haptic device 516 to provide primary haptic feedback (e.g., a puff or vortex of air, or an ultrasound beam) directed at the detected object location. If the user input 510 includes a secondary sensor 514, the user input 510 can be further configured to detect further insertion of the object at the secondary sensor 514, and to cause the haptic device 516 to provide secondary haptic feedback (e.g., a more aggressive level of haptic feedback) that induces withdrawal of the object from the user input 510 or indicates to the user that further movement of the object along the axis of travel is unnecessary or redundant.

The visual indicators 520 may include one or more light sources such as LEDs or other light emitting structures. Each visual indicator 520 may be associated with an individual user input 510 and may be located within or near the associated user input 510. For example, the visual indicator 520 may be a light ring disposed around an aperture of the associated user input 510, or may include one or more lights disposed on a faceplate 112 of the touchless control panel 500 adjacent to the user input 510 and/or an associated user input label 130 (as shown in FIG. 2A). Each individual visual indicator 520 may be activated by an activation signal transmitted from the processor 540, for example, in response to an input detected at the corresponding user input 510. The processor 540 can be further configured to deactivate each visual indicator 520 by transmitting a deactivation signal when appropriate (e.g., when a selected floor is reached, when an elevator arrives in response to a call button selection, etc.).

The audio indicator 570 may include a speaker or other device configured to produce a sound under control of the processor 540. The processor 540 may cause the audio indicator 570 to emit a sound based on an event such as a detection of an input at the primary sensor 512 of a user input 510. For example, when an input such as a floor selection is received at one of the user inputs 510, the processor 540 can cause the audio indicator 570 to play a sound indicating to the user that the input has been received. In some embodiments, the audio indicator 570 may be activated in conjunction with the visual indicators 520. For example, the processor 540 may cause the audio indicator 570 to play a sound at approximately the same time the processor 540 causes the visual indicator associated with the activated user input 510 to light up.

The disinfecting devices 530 may be, for example, the sterilizing light sources 122 configured to emit far-UVC light as described above with reference to FIG. 4, or any other suitable disinfecting device. For example, in some embodiments the disinfecting device 530 may be a spray device configured to spray a disinfecting or germicidal agent onto a surface of the touchless control panel 500. The disinfecting devices 530 may be configured for continuous operation, or may be activated by a signal from the processor 540 on a periodic or event-based schedule.

The processor 540 controls components of the touchless control panel 500 and may execute computer-executable instructions stored in the memory 550 or another non-transitory computer-readable medium. The processor may further be in communication with a communication interface 560. The communication interface may include wired or wireless connections to one or more external devices, such as other computing devices associated with the control functions of the user input 510. Where the touchless control panel 500 is an elevator control station or elevator call button panel, the communication interface 560 may be in communication with the elevator control circuitry that controls the operation of the elevator or elevators associated with the touchless control panel 500. For example, when a user inserts a finger into a user input 510 to select a floor, the processor 540 transmits a control signal via the communication interface 560 to control circuitry of the elevator indicating that the floor has been selected. The control signal may subsequently cause the elevator control circuitry to add the selected floor to a set of selected floors, cause the elevator to travel to the selected floor, etc., in accordance with any known automatic elevator control scheme.

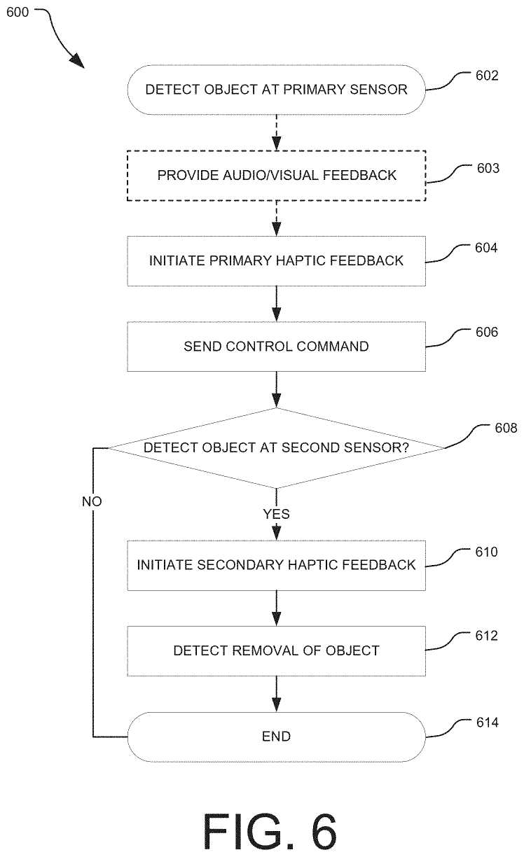

FIG. 6 is a flowchart illustrating an example method 600 of operating a touchless control panel in accordance with the present technology. The method 600 may be performed, for example, by one or more computer components of any of the touchless control panels 100, 500 described herein. Throughout the description of the method 600, reference will be made to components depicted in FIGS. 1-5 for exemplary illustrative purposes.

The method 600 begins at block 602 when an object is detected at a primary sensor 140/145, 512. For example, a photoelectric sensor or any other type of proximity sensor may detect that a finger or other object has been inserted into an aperture 120 of a user input 510 of the touchless control panel 100, 500. The inserted object triggers the primary sensor 140/145, 512 and causes the primary sensor 140/145, 512 to generate a first signal indicative of the detected object. When an object has been detected at the primary sensor, the method 600 continues to blocks 604 and 606. Optionally, at block 603, audio and/or visual feedback may be provided as well, as described elsewhere herein. In various embodiments, blocks 603, 604, and 606 may occur simultaneously, block 604 may occur before block 606, block 606 may occur before block 604, and block 603 may occur before or after either or both of blocks 604 and 606.

At block 604, the touchless control panel 100, 500 initiates primary haptic feedback in response to the detected object. For example, the processor 540 may cause the haptic device 160, 516 of the same user input 510 to direct a puff or vortex of air, or an ultrasound beam, toward the location of the object (e.g., toward the aperture 120). The primary haptic feedback indicates to the user that the input has been detected by the touchless control panel 100, 500.

At block 606, the touchless control panel 100, 500 sends a control signal 606 corresponding to the individual user input 510 at which the object was detected. For example, the processor 540 may send the control signal via the communication interface 560. In the exemplary implementation of an elevator control station, the processor 540 may send the control signal to an elevator control system or other computing device configured to control the movement and operation of the elevator. When primary haptic feedback has been initiated and the control signal has been sent, the method 600 continues to decision state 608.

At decision state 608, it is determined whether the objected is detected at a secondary sensor 150/155, 514. For example, a photoelectric sensor or any other type of proximity sensor may be disposed to detect when a finger or other object has been inserted further into the aperture 120 and is getting too close (e.g. has reached a predetermined minimum distance) to an interior surface of the touchless control panel 100, 500. The inserted object triggers the secondary sensor 150/155, 514 and causes the secondary sensor 150/155, 514 to generate a second signal indicative of the detected object. If an object is not detected at decision state 608, the method ends at block 614. If an object is detected at the secondary sensor, the method 600 continues to block 610.

At block 610, the touchless control panel 100, 500 initiates secondary haptic feedback in response to the detected object at the secondary sensor 150/155, 514. For example, the processor 540 may cause the haptic device 160, 516 of the same user input 510 to increase an intensity or frequency of the puff or vortex of air, or the ultrasound beam. The secondary haptic feedback indicates to the user that the inserted object has gone too far and should be removed. In some embodiments, the secondary haptic feedback has a heightened intensity or frequency sufficient to induce mild, non-harmful discomfort that causes the user to remove the finger or other object from the aperture 120. The secondary haptic feedback at block 610 may be maintained as long as the secondary sensor 150/155, 514 remains triggered (e.g., as long as the beam 152 is not detected at receiver 155).

At block 612, removal of the object is detected. For example, the removal of the object may cause the beam 152 to again reach the receiver 155 of the secondary sensor 150/155. The detection of the beam 152 at the receiver 155 may cause the secondary sensor 150/155, 514 to return to a non-triggered state. In response, the processor 540 may cause the haptic device 160 to either return to the primary haptic feedback level, or to cease providing haptic feedback. In some embodiments, the primary haptic feedback level may be maintained until the object is removed entirely and the beam 142 of the primary sensor 140/145 is again detected at the receiver 145. The method 600 ends at block 614, as the touchless control panel 100, 500 returns to a ready state for receiving a subsequent input from a user (e.g., to the ready state illustrated in FIG. 3A). If the touchless control panel 100, 500 includes a disinfecting device 530 that operates on an event-based schedule, block 614 may further include activating the disinfecting device 530 (such as a far-UVC LED or the like) to disinfect the user input 510.

### ADDITIONAL EMBODIMENTS

It will be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein. Although certain embodiments disclosed herein are described in the context of elevator control interfaces, the systems, devices, and methods described herein may equally be implemented in any other user interface context without departing from the spirit or scope of the present technology.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

1. A touchless control panel comprising:
a housing;
one or more user inputs, each of the one or more user inputs comprising:
an aperture extending through the housing;
a primary sensor disposed to detect objects inserted a first distance into the housing through the aperture, the primary sensor comprising a transmitter configured to emit a laser beam across at least a portion of the aperture and a receiver positioned to detect the laser beam, the primary sensor configured to generate a first signal when an object interrupts the laser beam; and
a haptic device configured to provide haptic feedback to objects inserted through the aperture; and
processing circuitry in communication with the one or more user inputs, the processing circuitry configured to, for each of the one or more user inputs:
receive the first signal from the primary sensor;
transmit a control signal based on the first signal; and
cause the haptic device to provide haptic feedback based on the first signal.

2. The touchless control panel of claim 1, wherein the haptic device comprises at least one of an ultrasound emitter or an air emitter at least partially disposed within the housing.

3. The touchless control panel of claim 1, wherein each of the one or more user inputs further comprises a secondary sensor disposed to detect objects inserted a second distance greater than the first distance into the housing through the aperture and to generate a second signal when an object is detected, the processing circuitry further configured to, for each of the one or more user inputs:
receive the second signal from the secondary sensor; and
cause the haptic device to provide an increased level of haptic feedback in response to the second signal.

4. The touchless control panel of claim 3, wherein the processing circuitry is further configured to cause the haptic device to discontinue the increased level of haptic feedback in response to a non-triggered state of the secondary sensor.

5. The touchless control panel of claim 4, wherein discontinuing the increased level of haptic feedback comprises returning to a reduced level of haptic feedback.

6. The touchless control panel of claim 4, wherein discontinuing the increased level of haptic feedback comprises deactivating the haptic device.

7. The touchless control panel of claim 1, wherein the transmitter is configured to emit the laser beam across a portion of an interior of the housing proximate the aperture.

8. The touchless control panel of claim 1, wherein each of the one or more user inputs further comprises a light source disposed to irradiate at least a portion of the housing with germicidal radiation.

9. The touchless control panel of claim 6, wherein the light germicidal radiation comprises ultraviolet light having a wavelength in a far-ultraviolet C wavelength range between about 207 nm and about 222 nm.

10. The touchless control panel of claim 6, wherein the light source comprises a ring extending along a lateral surface of the aperture, the ring comprising at least one visible light-emitting diode (LED) configured to emit visible light and at least one ultraviolet LED configured to emit far-ultraviolet C germicidal radiation.

11. The touchless control panel of claim 8, wherein the at least one ultraviolet LED is configured to operate continuously, and wherein the processing circuitry activates the at least one visible LED in response to the first signal.

12. The touchless control panel of claim 1, wherein the touchless control panel comprises at least one of an elevator control station or an elevator call button panel, and wherein the processing circuitry transmits the control signal to an elevator controller remote from the touchless control panel.

13. A method of operating a touchless control panel, the method comprising:
detecting an object at a first proximity sensor disposed within a housing of a touchless control panel; and
in response to detecting the object:
causing a haptic device within the housing to emit a gas toward the object to provide haptic feedback to the object; and
transmitting a control signal corresponding to a control function associated with the first proximity sensor.

14. The method of claim 13, wherein causing the haptic device to provide haptic feedback comprises causing the haptic device to emit compressed air.

15. The method of claim 13, further comprising:
detecting the object at a second proximity sensor disposed within the housing; and
in response to detecting the object at the second proximity sensor, causing the haptic device to increase an intensity or a frequency of the haptic feedback.

16. The method of claim 15, further comprising:
detecting, at the second proximity sensor, that the object has been at least partially removed; and
in response to detecting that the object has been at least partially removed, discontinuing the increased intensity or frequency of the haptic feedback.

17. The method of claim 16, wherein discontinuing the increased intensity or feedback comprises returning to a primary intensity or frequency until the object is removed from the housing.

18. The method of claim 16, wherein discontinuing the increased intensity or feedback comprises deactivating the haptic device.

19. The method of claim 13, further comprising, in response to detecting the object, providing a visual indication associated with the control function.

20. The method of claim 13, wherein the control signal causes, at least in part, an elevator controller to control an elevator to implement the control function.
